(19) United States
(12) Patent Application Publication
(10) Pub. No.: US 2023/0005602 A1
(43) Pub. Date: Jan. 5, 2023

(54) METHODS, SYSTEMS, AND COMPUTER PROGRAM PRODUCTS FOR DYNAMIC CACHING AND ROUTING OF HEALTH CARE PAYMENT PLAN ELIGIBILITY RESPONSES

(71) Applicant: Change Healthcare Holdings LLC, Nashville, TN (US)

(72) Inventors: Arien Malec, Oakland, CA (US); Peter Gott, Frisco, TX (US); Steven Martin, Kirkland, WA (US)

(21) Appl. No.: 17/363,781

(22) Filed: Jun. 30, 2021

(51) Int. Cl. G16H 40/20; G06Q 40/08; G06F 16/2455; G06N 5/02

(52) U.S. Cl. CPC G16H 40/20; G06Q 40/08; G06F 16/2455; G06N 5/02; G06Q 50/22

(57) ABSTRACT

A method includes maintaining a cache of past responses from a payor to past patient eligibility confirmation requests for one or more payment plans generated by a health care service provider; receiving a present patient eligibility confirmation request from the health care service provider to determine whether a patient is eligible for a payment plan of the one or more payment plans offered by a payor; querying the cache based on demographic information associated with the patient to determine whether the cache has eligibility information for the payment plan for the patient stored therein; selecting a portion of the eligibility information for the payment plan based on health care service provider preferences or payor preferences when the cache has the eligibility information for the payment plan for the patient stored therein; an returning the portion of the eligibility information to the health care service provider.

### FIELD

The present inventive concepts relate generally to health care systems and services and, more particularly, to management of health care payment plan eligibility responses.

### BACKGROUND

A health care service provider may query a health care payment plan administrator, e.g., a private insurance entity, government insurance entity, and/or a medical expense sharing organization, which may be referred to as a “payor,” to determine a patient's eligibility under a payment plan or coverage plan offered by the payor. This eligibility query may be performed at various stages during the patient care process, such as, for example, in advance of a patient's appointment, when the patient arrives for the appointment, and/or when generating a bill after a patient has been cared for by a health care service provider. The payment plan or coverage plan eligibility determination is used to ensure that the patient is billed correctly and receives all the benefits that the patient is entitled to. A payor may, however, receive high volumes of eligibility confirmation requests from health care service providers for the patients that they serve, which can overwhelm a payor's response infrastructure. As a result, health care service providers may not get timely eligibility information from a payor, which may result in billing errors and/or delays in submitting invoices to patients and/or the payor.

### SUMMARY

According to some embodiments of the inventive concept, a method comprises: maintaining a cache of past responses from a payor to past patient eligibility confirmation requests for one or more payment plans generated by a health care service provider; receiving a present patient eligibility confirmation request from the health care service provider to determine whether a patient is eligible for a payment plan of the one or more payment plans offered by a payor; querying the cache based on demographic information associated with the patient to determine whether the cache has eligibility information for the payment plan for the patient stored therein; selecting a portion of the eligibility information for the payment plan based on health care service provider preferences or payor preferences when the cache has the eligibility information for the payment plan for the patient stored therein; and returning the portion of the eligibility information to the health care service provider.

In other embodiments, querying the cache comprises determining whether to query the cache based on defined cache access rules.

In still other embodiments, returning the portion of the eligibility information comprises: determining whether to return the portion of the eligibility information based on defined cache information usage rules. The cache access rules and the cache information usage rules are provided by the health care service provider and/or the payor.

In still other embodiments, each of the past responses in the cache has a time-to-live (TTL) associated therewith.

In still other embodiments, the TTL is based on input received from the payor.

In still other embodiments, the method further comprises: determining that the payor is unavailable to provide a response to the present confirmation request from the health care service provider.

In still other embodiments, querying the cache comprises querying the cache responsive to determining that the payor is unavailable.

In still other embodiments, determining that the payor is unavailable to provide the response comprises: failing to receive responses to a plurality of patient eligibility confirmation requests for the one or more payment plans, respectively, which were forwarded to the payor over a time interval preceding receipt of the present patient eligibility confirmation request.

In still other embodiments, the method further comprises: receiving input from the payor that the payor is unavailable to provide a response to the present confirmation request from the health care service provider unless the cache does not have the eligibility information for the payment plan for the patient stored therein; and forwarding the present patient eligibility confirmation request to the payor responsive to determining that the cache does not have eligibility information for the payment plan for the patient stored therein.

In still other embodiments, maintaining the cache comprises maintaining the cache as a Bloom filter data structure.

In still other embodiments, querying the cache based on the demographic information comprises: forming a hash digest by combining portions of the demographic information; and querying the cache using the hash digest.

In still other embodiments, querying the cache based on the demographic information comprises: reducing a specificity of a portion of the demographic information; combining the portion of the demographic information with reduced specificity with at least one other portion of the demographic information to form a fuzzy hash digest; and querying the cache using the fuzzy hash digest.

In still other embodiments, the portion of the eligibility information comprises an indicium whether the patient is eligible for the payment plan.

In still other embodiments, returning the portion of the eligibility information to the health care service provider comprises returning the indicum to the health care service provider only when the indicium specifies the patient is not eligible for the payment plan. The method further comprises: forwarding the present patient eligibility confirmation request to the payor when the indicium specifies the patient is eligible for the payment plan.

In still other embodiments, returning the portion of the eligibility information to the health care service provider comprises returning the indicum to the health care service provider only when the indicium specifies the patient is eligible for the payment plan. The method further comprises: forwarding the present patient eligibility confirmation request to the payor when the indicium specifies the patient is not eligible for the payment plan.

In still other embodiments, the payment plan is a first payment plan. The portion of the eligibility information comprises information that the patient is eligible for a second payment plan.

In still other embodiments, the one or more payment plans offered by the payor include the second payment plan.

In still other embodiments, the second payment plan is offered by a second payor different than the first payor.

In still other embodiments, the first payor is a private entity and the second payor is a governmental entity or the first payor is the governmental entity and the second payor is the private entity.

In still other embodiments, the patient is eligible for the second payment plan based on a familial relationship.

In still other embodiments, the method further comprises using an Artificial Intelligence (AI) prediction engine to predict when the present patient eligibility confirmation request is received from the health care service provider. Querying the cache comprises: querying the cache based on identifying information for the health care service provider to obtain payment plan eligibility information for all patients served by the health care service provider prior to receiving the present patient eligibility confirmation request; and searching the payment plan eligibility information for all patients served by the health care service provider based on the demographic information associated with the patient to determine whether the cache had eligibility information for the payment plan for the patient stored therein.

In some embodiments of the inventive concept, a system comprises: a processor; and a memory coupled to the processor and comprising computer readable program code embodied in the memory that is executable by the processor to perform operations comprising: maintaining a cache of past responses from a payor to past patient eligibility confirmation requests for one or more payment plans generated by a health care service provider; receiving a present patient eligibility confirmation request from the health care service provider to determine whether a patient is eligible for a payment plan of the one or more payment plans offered by a payor; querying the cache based on demographic information associated with the patient to determine whether the cache has eligibility information for the payment plan for the patient stored therein; selecting a portion of the eligibility information for the payment plan based on health care service provider preferences or payor preferences when the cache has the eligibility information for the payment plan for the patient stored therein; and returning the portion of the eligibility information to the health care service provider.

In still other embodiments of the inventive concept, a computer program product, comprises: a non-transitory computer readable storage medium comprising computer readable program code embodied in the medium that is executable by a processor to perform operations comprising: maintaining a cache of past responses from a payor to past patient eligibility confirmation requests for one or more payment plans generated by a health care service provider; receiving a present patient eligibility confirmation request from the health care service provider to determine whether a patient is eligible for a payment plan of the one or more payment plans offered by a payor; querying the cache based on demographic information associated with the patient to determine whether the cache has eligibility information for the payment plan for the patient stored therein; selecting a portion of the eligibility information for the payment plan based on health care service provider preferences or payor preferences when the cache has the eligibility information for the payment plan for the patient stored therein; and returning the portion of the eligibility information to the health care service provider.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims. It is further intended that all embodiments disclosed herein can be implemented separately or combined in any way and/or combination.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a system for dynamic caching and routing of health care payment plan eligibility responses in accordance with some embodiments of the inventive concept;

FIG. 2 is a message flow diagram illustrating operations for dynamic caching and routing of health care payment plan eligibility responses in accordance with some embodiments of the inventive concept;

FIGS. 3-6 are flowcharts that illustrate operations for dynamic caching and routing of health care payment plan eligibility responses in accordance with some embodiments of the inventive concept;

FIG. 7 is a data processing system that may be used to implement one or more servers in the system for dynamic caching and routing of health care payment plan eligibility responses of FIG. 1 in accordance with some embodiments of the inventive concept; and

FIG. 8 is a block diagram that illustrates a software/hardware architecture for use in the system for dynamic caching and routing of health care payment plan eligibility responses of FIG. 1 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components, and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, a payor is an entity that provides payment in whole or in part on behalf of a patient that receives health care services from a health care service provider. A payor may include, but is not limited to, a private insurance entity, a governmental insurance entity, and/or a medical expense sharing entity. Health care services may include, but are not limited to, interactive examinations, procedures, laboratory tests, prescriptions, and the like.

Some embodiments of the inventive concept stem from a realization that a payor's computer system for responding to payment plan or coverage eligibility requests from health care service providers for their patients can get overwhelmed resulting in the health care service providers getting error messages or timeouts in response to their requests. As a result, health care service providers may not be able to verify the payment plan coverage status of their patients in a timely manner, which can result in delays and/or errors in billing patients and/or their payors. Some embodiments of the inventive concept may provide an eligibility management system for assisting a payor in processing patient payment plan eligibility requests that are generated by health care service providers. The eligibility management system may act as an intermediary between the health care service providers and payors by monitoring past responses from payors to patient eligibility confirmation requests for one or more payment plans associated with the payors that are generated by health care service providers. The system may maintain a cache based on the history of responses from the payors for the various patients served by the health care service providers. When a payor's response infrastructure is operating at a functional level that it is able to handle the number of incoming patient eligibility confirmation requests, then the eligibility management system may route these requests to the payor or one of the payor's intermediaries for generating the responses for the health care service providers. When a payor's response infrastructure is out of service or is generating time-outs due to the number of eligibility requests exceeding its capacity, the eligibility response management system may intervene and rather than route the incoming requests to the payor or one of the payors intermediaries, the eligibility response management system may query the cache to determine that previous responses have been generated for the particular patients associated with the requests. If a previous response has been generated for a particular patient, then the eligibility management system may proceed in a variety of different ways based on rules that express payor and/or service provider preferences. That is, in some embodiments, the payors and/or the health care service providers may communicate with the eligibility management system to define rules that express their preferences in handling responses for eligibility confirmation requests when the payor is unable to generate a response directly. The rules may specify, for example, whether/when to access the cache, whether/when to use the results of the cache if/when the cache is accessed, and/or what portions of information obtained from the cache may be used when the cache is accessed. For example, a payor and/or a health care service provider may define a variety of rules including, but not limited to the following examples: rules that specify a time-to-live (TTL) for responses stored in the cache on a per payor basis; rules that define circumstances on whether a cached eligibility response is returned to the health care service provider, e.g., only return a cached ineligible response, only return a cached eligible response, return cached eligible and ineligible responses; rules that define how much additional information may be returned to the health care service provider from the cached eligibility response, such as benefits details, co-pay amounts, deductible caps, and the like; rules that specify a particular age limit on cached information that may be different from a TTL value, such as only return information from a cached response if the cached response does not exceed a specified age; rules that provide for discovery of additional coverage for a patient, such as detecting that a patient is also covered by one or more additional payment or coverage plans associated with one or more payors including private payors and/or governmental payors due to, for example, a familial relationship; and rules that define when the eligibility management system intervenes to generate a response for a health care service provider based on the cached responses, e.g., a payor may define a rule that a response should always be generated from the cache unless the cache does not have any response information for a particular patient (cache miss), a response should only be generated from the cache after the payor has generated time-outs or errors for a particular time period, or never use the cache for a particular payor or health care service provider.

Thus, an eligibility response management system, according to some embodiments of the inventive concept, may provide dynamic caching and routing functionality for managing patient eligibility confirmation requests that can be customized using a control plane to reflect preferences or requirements associated with the payors and/or the health care service providers.

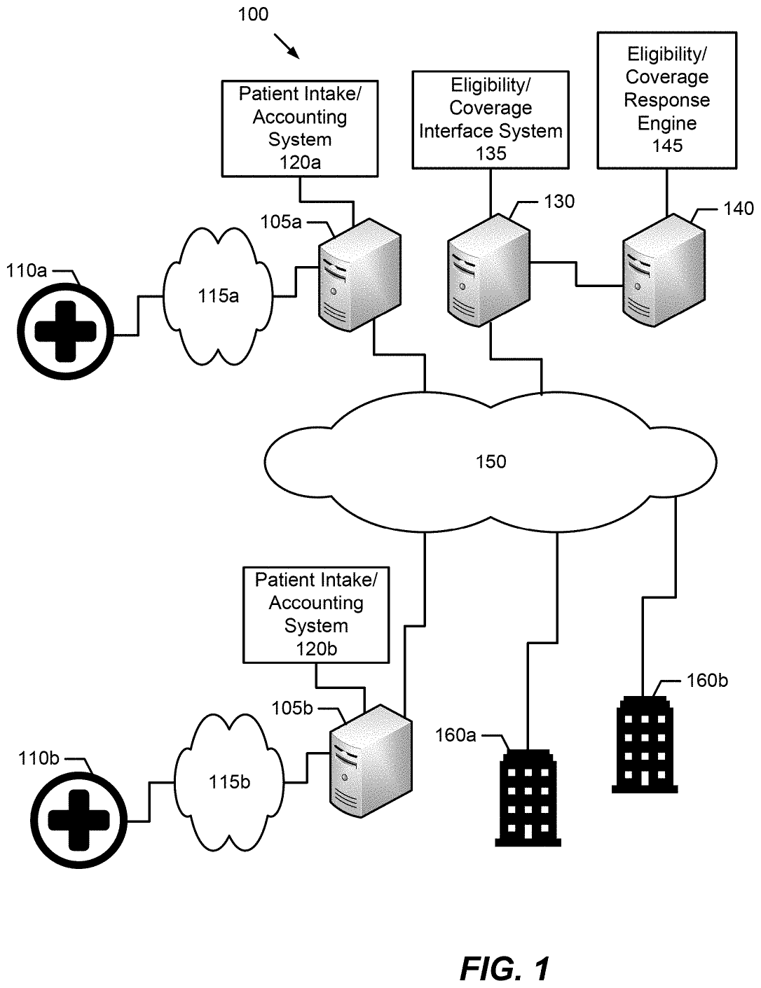

Referring to FIG. 1, a communication network 100 including an eligibility management system for dynamic caching and routing of health care plan eligibility responses, in accordance with some embodiments of the inventive concept, comprises multiple health care provider facilities or practices 110a, 110b. Each health care provider facility or practice may represent various types of organizations that are used to deliver health care services to patients via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, private insurance plans, government insurance plans (e.g., Medicare, Medicaid, state, or federal public employee insurance plans), hybrid insurance plans (e.g., Affordable Care Act plans), private medical cost sharing plans, and the patients themselves. Two provider facilities 110a, 110b are illustrated in FIG. 1 with the first provider including a first patient intake/accounting system server 105a accessible via a network 115a. The first patient intake/accounting system server 105a may be configured with a patient intake/accounting system module 120a to manage the intake of patients for appointments and to generate invoices for payors for services and products rendered through the provider 110a. The patient intake/accounting system 120a may be configured to perform payment plan eligibility confirmation to ensure that a patient is covered by a particular payment plan. The patient intake/accounting system 120a may generate a patient eligibility confirmation request for a payor at various times when serving a patient. For example, an eligibility confirmation request may be generated prior to a patient's appointment. Some health care service providers, for example, generate eligibility confirmation requests for all patients that will be seen in a particular week or other time period. An eligibility confirmation request may also be generated when a patient arrives for an appointment. An eligibility confirmation request may also be generated when a health care service provider generates an invoice for services and products rendered in caring for a patient. The network 115a communicatively couples the first patient intake/accounting system server 105a to other devices, terminals, and systems in the provider's facility 110a. The network 115a may comprise one or more local or wireless networks to communicate with first patient intake/accounting system server 105a when the first patient intake/accounting system server 105a is located in or proximate to the health care service provider facility 110a. When the first patient intake/accounting system server 105a is in a remote location from the health care facility, such as part of a cloud computing system or at a central computing center, then the network 115a may include one or more wide area or global networks, such as the Internet. The second provider facility 110b is similar to the first provider facility 110a and includes a second patient intake/accounting system server 105b, which is configured with a patient intake/accounting system server 120b. The second patient intake/accounting system server 105b is coupled to other devices, terminals, and systems in the provider's facility 110b via a network 115b.

According to embodiments of the inventive concept, a system may use an intermediary between health care service providers and payors for managing patient eligibility confirmation requests. Moreover, these requests can be managed in a customized way for the health care service providers and the payors using a control plane to reflect preferences or requirements associated with the payors and/or the health care service providers. The eligibility management system may include an eligibility management interface server 130, which includes an eligibility/coverage interface system module 135, and an eligibility/coverage response engine server 140, which includes an eligibility/coverage response engine 145. The eligibility/coverage interface system module 135 may be configured to receive eligibility confirmation requests from one or more health care service providers 110a, 110b, to communicate with the appropriate payor 160a, 160b and/or the eligibility/coverage response engine server 140 to obtain responses for the requests based on service provider and/or payor preferences, and to communicate a response back to the health care service providers 110a,110b by way of the patient intake/accounting systems 120a, 120b based on the service provider and/or the payor preferences. The eligibility/coverage response engine module 145 may be configured to maintain a cache containing responses to patient eligibility confirmation requests from payors, such as payors 160a, 160b, to route incoming patient eligibility confirmation requests to the appropriate entity, e.g., a payor 160a, 160b, a payors intermediary, and/or to process the request locally using cached response information. The eligibility/coverage response engine module 145 may be further configured to provide APIs for health care service providers 110a, 110b and/or payors 160a, 160b to all these entities to create rules expressing their preferences for how the cached responses may be used in responding to patient eligibility confirmation requests generated from the health care service providers 110a, 110b.

A network 150 couples the patient intake/accounting system servers 105a, 105b to the eligibility/coverage interface system server 130 and couples the payors 160a and 160b to the eligibility/coverage interface system server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The service provided through the eligibility/coverage interface system server 130, eligibility/coverage interface system module 135, eligibility/coverage response engine server 140, and eligibility/coverage response engine module 145 to provide an eligibility management system for dynamic caching and routing of health care plan eligibility responses may, in some embodiments, be embodied as a cloud service. For example, health care service providers and/or payors may access the eligibility management system service as a Web service. In some embodiments, the eligibility management system service may be implemented as a Representational State Transfer Web Service (RESTful Web service).

Although FIG. 1 illustrates an example communication network including an eligibility management system for dynamic caching and routing of health care plan eligibility responses, it will be understood that embodiments of the inventive subject matter are not limited to such configurations, but are intended to encompass any configuration capable of carrying out the operations described herein.

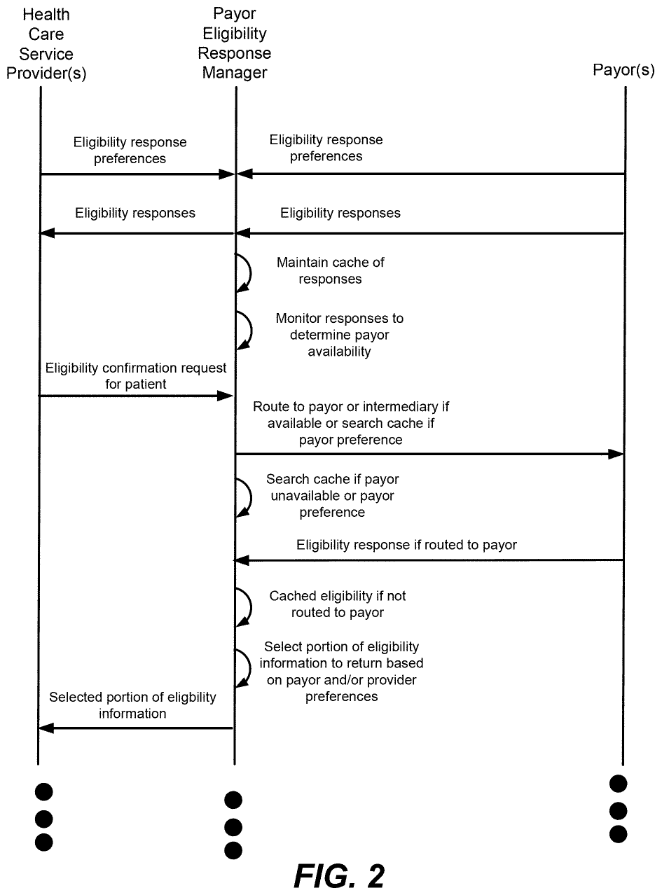

FIG. 2 is a message flow diagram illustrating operations for dynamic caching and routing of health care payment plan eligibility responses in accordance with some embodiments of the inventive concept. As shown in FIG. 2, health care service providers 110a, 110b and payors 160a, 160b may communicate with a payor eligibility response manager, such as an eligibility management system embodied by the eligibility/coverage interface system server 130, eligibility/coverage interface system module 135, eligibility/coverage response engine server 140, and eligibility/coverage response engine module 145 of FIG. 1. The payor eligibility response manager may provide APIs, which can be used by the health care service provider(s) and the payor(s) to express their preferences for how cached responses may be used in responding to patient eligibility confirmation requests through creation of rules and guidelines. The payor eligibility response manager may monitor ongoing patient eligibility responses from payor(s) to health care service provider(s) and may maintain these responses in a cache, subject to TTL rules established by the health care service provider(s) and/or the payor(s). The payor eligibility response manager may also monitor payor responses to assess the payor's availability. For example, if a payor has generated a specified number of error messages or time-outs within a specified time interval, then it may be concluded that the payor's system is unavailable to process patient eligibility confirmation requests at this time. When a health care service provider transmits an eligibility confirmation request for a patient to the payor eligibility response manager, the payor eligibility response manager may route the request to the payor or payor's intermediary or search the cache based on payor preference. As described above, even if the payor is available, the payor may use the payor eligibility response manager to use cached responses in response to eligibility requests with requests only being forwarded to the payor when there is a cache miss. Such a rule may be further subject to preferences or requirements of a health care service provider as a provider may place limits on when the use of cached responses is acceptable. The payor eligibility response manager may resolve conflicts between rules using a variety of mechanisms including defaulting to the most restrictive rule. Thus, if the payor is unavailable or if the payor has a preference for use of the cache even when available, then the payor eligibility response manager will search the cache for a response corresponding to the particular patient and payor. The payor eligibility response manager may then obtain either a cached eligibility response (if available through a cache hit) or an eligibility response from the payor if the request was routed to the payor. The payor eligibility manager may then select a portion of the eligibility information obtained through the response to return to the health care service provider. The portion selected and the nature of the returned information may be based on health care service provider and/or payor preferences expressed through the rules created by way of the APIs. As described above, a variety of types and amounts of information may be returned to the health care service provider based on the defined preferences. For example, for cached responses, a rule may specify that a response should be returned only if the patient is eligible or only if the patient is ineligible. A more expansive rule, for example, may allow the payor eligibility response manager to return a response based on cached information for both eligible and ineligible patients, but additional benefits information may not be included. Less restrictive rules may allow the payor eligibility response manager to provide benefits information based on cached response information in addition to payment plan eligibility status. Even less restrictive rules may allow the payor eligibility response manager to construct a broader picture of a patient's health care coverage by including potential coverage options with the same or different payor based on, for example, the patient's relationship with other people, such as a family member, through which the patient may access coverage. This information may be provided to the health care service provider in addition to the eligibility confirmation requested. Such rules may be applied in the same or different ways based on responses received from the payor in conjunction with or independent from the cached responses.

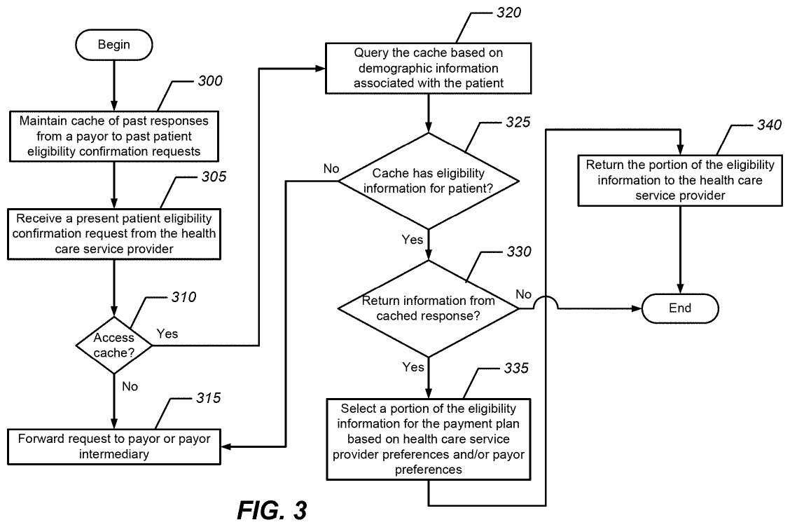

FIGS. 3-6 are flowcharts that illustrate operations for dynamic caching and routing of health care payment plan eligibility responses in accordance with some embodiments of the inventive concept. The example operations are described with respect to a single health care service provider submitting a patient eligibility confirmation request for a single patient to a single payor. It will be understood that the eligibility management system described herein and the operations associated therewith are applicable to managing patient eligibility confirmation requests for multiple payors that were generated by multiple health care service providers serving multiple patients. Referring now to FIG. 3, a cache is maintained of past responses from a payor to past patient eligibility confirmation requests for one or more payment plans generated by a health care service provider (block 300). A present patient eligibility confirmation request from the health care service provider is received (block 305) and, based on rules established by the health care service provider and/or the payor, a determination is made whether to access the cache (block 310). Payors and/or health care service providers may define a variety of different rules for when the cache is accessed. For example, a payor may define a rule that the cache should be accessed and a response generated therefrom unless there is a cache miss, i.e., the cache does not have previous eligibility information stored for a particular patient. In other embodiments, rules may be defined that specify that a response should be generated from the cache only after the number of time-outs or errors received from a payor in a defined time period exceeds a defined threshold. In still other embodiments, rules may defined that the cache is not used for particular payors and/or health care service providers. If the cache is not accessed, then the request is forwarded to the payor or payor intermediary (block 315). Otherwise, the cache is queried (block 320) using demographic information associated with the patient to determine whether the cache has eligibility information for the payment plan for the patient stored therein. If the cache does not have eligibility information stored therein for the patient (block 325), i.e., a cache miss, then the request is forwarded to the payor or payor intermediary. (block 315). Otherwise, a determination is made whether to return information to the health care service provider based on defined cache information usage rules (block 330). Even if the cache contains eligibility information for the patient, rules may be defined by the payors and/or health care service providers that limit whether any information is returned and how much information is returned. For example, a payor and/or a health care service provider may define a time-to-live (TTL) for responses stored in the cache on a per payor basis such that eligibility information that is older than a certain age is not returned to a health care service provider. Other rules may be defined specifying that the eligibility response management system should only return a cached ineligible response, only return a cached eligible response, or return both cached eligible and ineligible responses. In some embodiments, additional patient information may be stored in the cache and rules may be defined that specify how much, if any, of that information is returned to the health care service provider. This additional information may include, but is not limited to benefits details, co-pay amounts, deductible caps, and the like. This additional information may have different TTL values associated therewith as compared to the TTL used for eligibility information. Rules may also be defined that provide for discovery of additional coverage for a patient, such as detecting that a patient is also covered by one or more additional payment or coverage plans associated with one or more payors including private payors and/or governmental payors due to, for example, a familial relationship. Based on the evaluations of the rules for returning information from the cached response for the patient (block 330), no information may be returned or some of all of the cached information may be returned (block 335). A portion of the eligibility information for the payment plan is selected (block 335) based on the health care service provider preferences or the payor preferences as expressed by the defined rules. This portion may be a subset of the cached information for the patient or all of the cached information for the patient. The selected portion of the eligibility information is then returned (block 340) to the health care service provider.

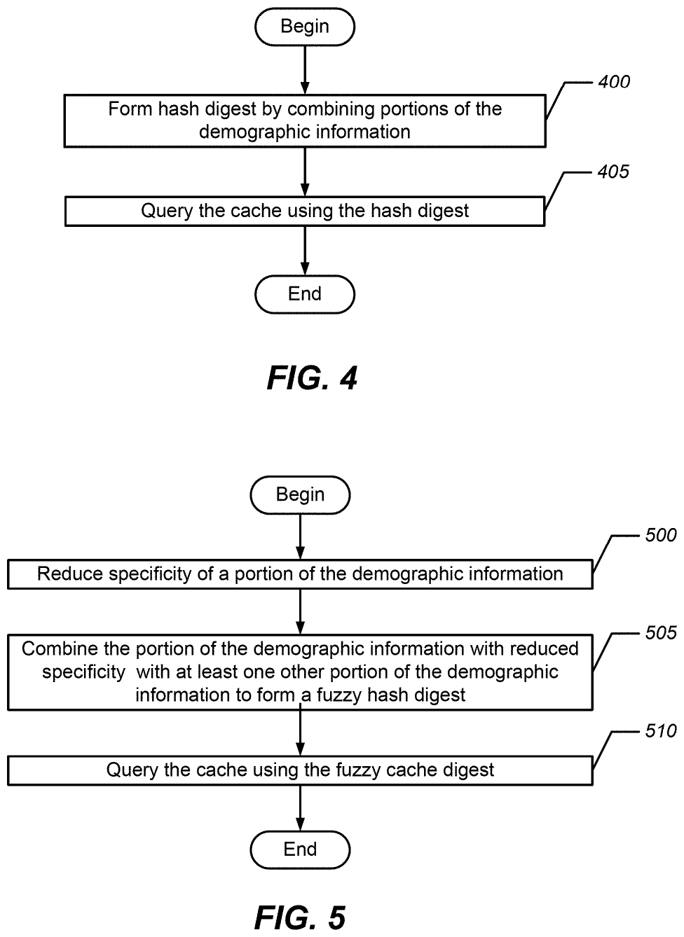

The cache of the payor responses to eligibility confirmation requests may be embodied in a variety of ways according to different embodiments of the inventive concept. In some embodiments, the cache may be implemented using a Bloom filter data structure. The Bloom filter data structure tells whether an element may be in a set or is definitely not in the set. The only possible errors are false positives. Thus, a search for a nonexistent element can give an incorrect answer. With more elements in the filter, the error rate increases. Referring now to FIG. 4, the cache may also be embodied through the use of a hash digest. A hash digest is formed by combining portions of the patient demographic information used as a hash key (block 400). The cache may then be queried using the hash digest formed through a combination of portions of the demographic information (block 405). In some embodiments, the hash digest approach may be further modified to use a fuzzy hash digest. Referring now to FIG. 5, the specificity of a portion of the patient demographic information may be reduced (block 500). The portion of the demographic information with reduced specificity is combined with at least one other portion of the demographic information to form a fuzzy hash digest (block 505). The fuzzy hash digest may then be used to query the cache (block 510). Examples of the use of a fuzzy hash digest for searching a cache are described, for example, in U.S. Pat. No. 10,510,440 the disclosure of which is incorporated herein by reference.

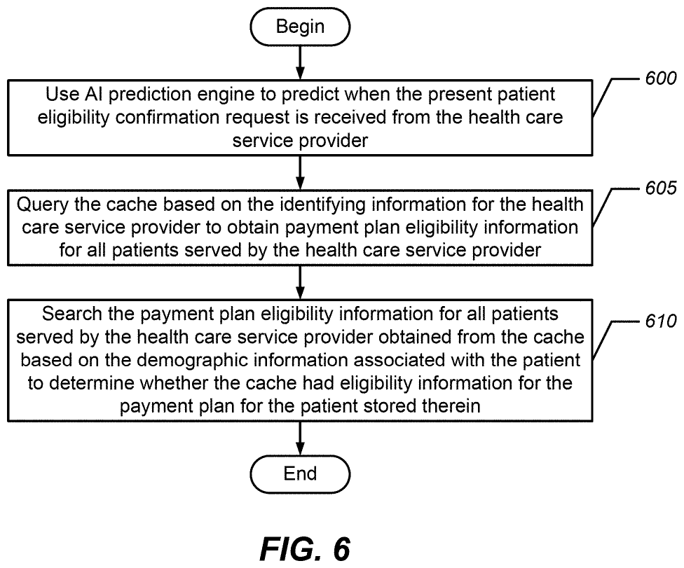

As described above, health care service providers may submit patient eligibility confirmation requests to payors on a regular basis, such as the beginning of every week for all patients with appointments that week. The eligibility management system may include an Artificial Intelligence (AI) prediction engine as part of the eligibility/coverage response engine module 145 that can be trained based on historical patterns of patient eligibility confirmation requests to predict arrival times for new patient eligibility confirmation requests, such as when batch requests are received for all patients to be seen by a health care service provider in an upcoming time period. The AI prediction engine may be embodied in a variety of ways including, but not limited to, a machine learning system, a deep learning system, and/or a multi-layer neural network. Moreover, it will be understood that the multi-layer neural network is a multi-layer artificial neural network comprising artificial neurons or nodes and does not include a biological neural network comprising real biological neurons. Referring now to FIG. 6, an AI prediction engine is used to predict when a present eligibility confirmation request will be received from the health care service provider (block 600). The cache is queried (block 605) based on the identifying information for the health care service provider to obtain payment plan eligibility information for all patients served by the health care service provider (block 605) prior to receiving the present patient eligibility confirmation request. Thus, the cache is queried in advance to obtain all the cached eligibility responses for a particular health care service provider. When the present patient eligibility confirmation request is received, the payment plan eligibility information for all of the patients served by the health care service provider obtained from the cache query at block 605 is searched (block 610) based on the demographic information for the patient to determine whether the cache had eligibility information for the payment plan for the patient stored therein. The use of an AI prediction engine may allow the cache queries to be performed during time intervals when processing demands are lower, which may improve the efficiency and responsiveness of the overall eligibility management system.

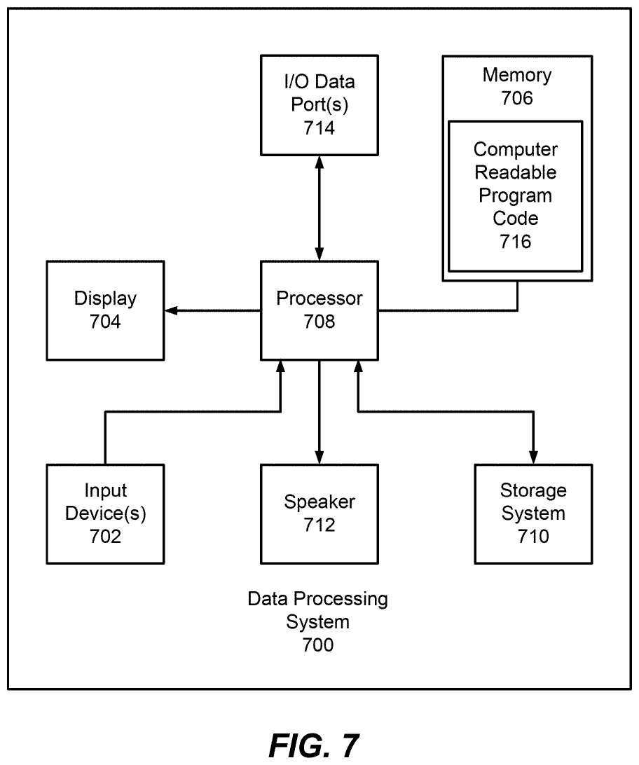

Referring now to FIG. 7, a data processing system 700 that may be used to implement the eligibility/coverage response engine server 140 of FIG. 1, in accordance with some embodiments of the inventive concept, comprises input device(s) 702, such as a keyboard or keypad, a display 704, and a memory 706 that communicate with a processor 708. The data processing system 700 may further include a storage system 710, a speaker 712, and an input/output (I/O) data port(s) 714 that also communicate with the processor 708. The processor 708 may be, for example, a commercially available or custom microprocessor. The storage system 810 may include removable and/or fixed media, such as floppy disks, ZIP drives, hard disks, or the like, as well as virtual storage, such as a RAMDISK. The I/O data port(s) 714 may be used to transfer information between the data processing system 700 and another computer system or a network (e.g., the Internet). These components may be conventional components, such as those used in many conventional computing devices, and their functionality, with respect to conventional operations, is generally known to those skilled in the art. The memory 706 may be configured with computer readable program code 716 to facilitate management of patient eligibility confirmation requests and responses thereto according to some embodiments of the inventive concept.

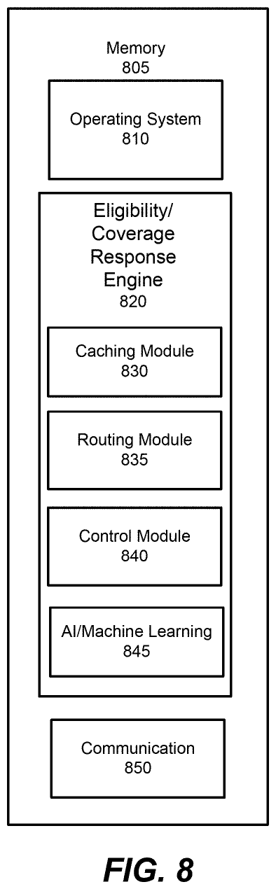

FIG. 8 illustrates a memory 805 that may be used in embodiments of data processing systems, such as the eligibility/coverage response engine server 140 of FIG. 1, the eligibility/coverage interface system server 130 of FIG. 1, and the data processing system 700 of FIG. 7, respectively, to facilitate management of patient eligibility confirmation requests and responses thereto according to some embodiments of the inventive concept. The memory 805 is representative of the one or more memory devices containing the software and data used for facilitating operations of the eligibility/coverage response engine server 140 and the eligibility/coverage response engine module 145 as described herein. The memory 805 may include, but is not limited to, the following types of devices: cache, ROM, PROM, EPROM, EEPROM, flash, SRAM, and DRAM. As shown in FIG. 8, the memory 805 may contain three or more categories of software and/or data: an operating system 810, an eligibility/coverage response engine module 820, and a communication module 850. In particular, the operating system 810 may manage the data processing system's software and/or hardware resources and may coordinate execution of programs by the processor. The eligibility/coverage response engine module 820 may include a caching module 830, a routing module 835, a control module 840, and an AI/machine learning module 845. The caching module 830 may be configured to perform one or more of the operations described above with respect to FIGS. 1-6 and which are related to maintaining and managing a cache of past responses from one or more payors to past patient eligibility confirmation requests generated by one or more health care service providers. The routing module 835 may be configured to perform one or more of the operations described above with respect to FIGS. 1-6 and which are related to routing a patient eligibility confirmation request to a payor or a payor's intermediaries or processing the patient eligibility confirmation request at the eligibility management system by querying a cache to search for a previously generated cached eligibility response for that patient. The control module 840 may be configured to perform one or more of the operations described above with respect to FIGS. 1-6 and which are related to providing one or more APIs to allow health care service providers and/or payors to create rules to express their preferences with respect to use of the cache for responding to patient eligibility confirmation requests and with respect to the quantity and type of information provided back to health care service providers in response patient eligibility confirmation requests. The AI/machine learning module is configured to perform one or more of the operations described above with respect to FIGS. 1-6 and which are related to predicting the arrival of patient eligibility confirmation request(s) from health care service providers to allow the cache to be queried in advance for patients associated with that health care service provider of the actual arrival of the patient eligibility confirmation request(s). The communication module 850 may be configured to support communication between, for example, the eligibility/coverage response engine server 140 and the eligibility/coverage interface system server 130, between the eligibility/coverage interface system server 130 and the patient intake/accounting system servers 105a, 105b, and between the eligibility/coverage interface system server 130 and the payors 160a, 160b.

Although FIGS. 7-8 illustrate hardware/software architectures that may be used in data processing systems, such as the eligibility/coverage response engine server 140 of FIG. 1, the eligibility/coverage interface system server 130, and the data processing system 700 of FIG. 7, respectively, in accordance with some embodiments of the inventive concept, it will be understood that embodiments of the present invention are not limited to such a configuration but is intended to encompass any configuration capable of carrying out operations described herein.

Computer program code for carrying out operations of data processing systems discussed above with respect to FIGS. 1-8 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the eligibility/coverage response engine server 140 of FIG. 1, the eligibility/coverage interface system server 130, and the data processing system 700 of FIG. 7 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-8 may be used to facilitate management of patient eligibility confirmation requests and responses thereto according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 805 when coupled to a processor includes computer readable program code that, when executed by the processor, causes the processor to perform operations including one or more of the operations described herein with respect to FIGS. 1-6.

Some embodiments of the inventive concept described herein may provide an eligibility management system using dynamic caching and routing of health care provider eligibility confirmation requests and payor responses. A payor may use the eligibility management system for assistance in processing patient eligibility confirmation requests from health care service providers when the payor's response infrastructure is available to reduce the load on the payor's response infrastructure and when the payor's response infrastructure is down or otherwise unavailable to respond. The eligibility management system may provide APIs that can be used by payors and/or health care service providers to control how the cache of payor responses is used and the quantity and type of information that is provided to a health care service provider from cached information and/or new responses received from a payor. Some embodiments may provide allow for the compilation of benefits and coverage scope for a patient across multiple plans and/or payors based on cached eligibility response information and/or current eligibility response from a payor. This information may be selectively provided to one or more health care providers, which may ensure a patient receives all the benefits to which the patient is entitled. An AI capability may be used to predict when eligibility confirmation requests may be received from a health care service provider allowing the eligibility management system to query the cache in advance at a time that reduces the impact on processor resources. Embodiments of an eligibility management system may, therefore, facilitate the processing of eligibility confirmation requests in a more dynamic and robust manner to improve assessments of a patient's eligibility for one or more payment plans or benefits provided by one or more payors. This may reduce errors and/or improve timeliness when health care service providers invoice payors and patients.

### Further Definitions and Embodiments

In the above description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
maintaining a cache of past responses from a payor to past patient eligibility confirmation requests for one or more payment plans generated by a health care service provider;
receiving a present patient eligibility confirmation request from the health care service provider to determine whether a patient is eligible for a payment plan of the one or more payment plans offered by a payor;
querying the cache based on demographic information associated with the patient to determine whether the cache has eligibility information for the payment plan for the patient stored therein;
selecting a portion of the eligibility information for the payment plan based on health care service provider preferences or payor preferences when the cache has the eligibility information for the payment plan for the patient stored therein; and
returning the portion of the eligibility information to the health care service provider.

2. The method of claim 1, wherein querying the cache comprises:
determining whether to query the cache based on defined cache access rules.

3. The method of claim 2, wherein returning the portion of the eligibility information comprises:
determining whether to return the portion of the eligibility information based on defined cache information usage rules;
wherein the cache access rules and the cache information usage rules are provided by the health care service provider and/or the payor.

4. The method of claim 1, wherein each of the past responses in the cache has a time-to-live (TTL) associated therewith.

5. The method of claim 4, wherein the TTL is based on input received from the payor.

6. The method of claim 1, further comprising:
determining that the payor is unavailable to provide a response to the present confirmation request from the health care service provider.

7. The method of claim 6, wherein querying the cache comprises querying the cache responsive to determining that the payor is unavailable.

8. The method of claim 6, wherein determining that the payor is unavailable to provide the response comprises:
failing to receive responses to a plurality of patient eligibility confirmation requests for the one or more payment plans, respectively, which were forwarded to the payor over a time interval preceding receipt of the present patient eligibility confirmation request.

9. The method of claim 1, further comprising:
receiving input from the payor that the payor is unavailable to provide a response to the present confirmation request from the health care service provider unless the cache does not have the eligibility information for the payment plan for the patient stored therein; and
forwarding the present patient eligibility confirmation request to the payor responsive to determining that the cache does not have eligibility information for the payment plan for the patient stored therein.

10. The method of claim 1, wherein querying the cache based on the demographic information comprises:
forming a hash digest by combining portions of the demographic information; and
querying the cache using the hash digest.

11. The method of claim 10, wherein querying the cache based on the demographic information comprises:
reducing a specificity of a portion of the demographic information;
combining the portion of the demographic information with reduced specificity with at least one other portion of the demographic information to form a fuzzy hash digest; and
querying the cache using the fuzzy hash digest.

12. The method of claim 1, wherein the portion of the eligibility information comprises an indicium whether the patient is eligible for the payment plan.

13. The method of claim 12, wherein returning the portion of the eligibility information to the health care service provider comprises returning the indicum to the health care service provider only when the indicium specifies the patient is not eligible for the payment plan;
wherein the method further comprises:
forwarding the present patient eligibility confirmation request to the payor when the indicium specifies the patient is eligible for the payment plan.

14. The method of claim 12, wherein returning the portion of the eligibility information to the health care service provider comprises returning the indicum to the health care service provider only when the indicium specifies the patient is eligible for the payment plan;
wherein the method further comprises:
forwarding the present patient eligibility confirmation request to the payor when the indicium specifies the patient is not eligible for the payment plan.

15. The method of claim 1, wherein the payment plan is a first payment plan; and
wherein the portion of the eligibility information comprises information that the patient is eligible for a second payment plan.

16. The method of claim 15, wherein the one or more payment plans offered by the payor include the second payment plan.

17. The method of claim 15, wherein the second payment plan is offered by a second payor different than the first payor.

18. The method of claim 15, wherein the patient is eligible for the second payment plan based on a familial relationship.

19. The method of claim 1, further comprising:
using an Artificial Intelligence (AI) prediction engine to predict when the present patient eligibility confirmation request is received from the health care service provider; and
wherein querying the cache comprises:
querying the cache based on identifying information for the health care service provider to obtain payment plan eligibility information for all patients served by the health care service provider prior to receiving the present patient eligibility confirmation request; and
searching the payment plan eligibility information for all patients served by the health care service provider based on the demographic information associated with the patient to determine whether the cache had eligibility information for the payment plan for the patient stored therein.

20. A system, comprising:
a processor; and
a memory coupled to the processor and comprising computer readable program code embodied in the memory that is executable by the processor to perform operations comprising:
maintaining a cache of past responses from a payor to past patient eligibility confirmation requests for one or more payment plans generated by a health care service provider;
receiving a present patient eligibility confirmation request from the health care service provider to determine whether a patient is eligible for a payment plan of the one or more payment plans offered by a payor;
querying the cache based on demographic information associated with the patient to determine whether the cache has eligibility information for the payment plan for the patient stored therein;
selecting a portion of the eligibility information for the payment plan based on health care service provider preferences or payor preferences when the cache has the eligibility information for the payment plan for the patient stored therein; and
returning the portion of the eligibility information to the health care service provider.

21. A computer program product, comprising:
a non-transitory computer readable storage medium comprising computer readable program code embodied in the medium that is executable by a processor to perform operations comprising:
maintaining a cache of past responses from a payor to past patient eligibility confirmation requests for one or more payment plans generated by a health care service provider;
receiving a present patient eligibility confirmation request from the health care service provider to determine whether a patient is eligible for a payment plan of the one or more payment plans offered by a payor;
querying the cache based on demographic information associated with the patient to determine whether the cache has eligibility information for the payment plan for the patient stored therein;
selecting a portion of the eligibility information for the payment plan based on health care service provider preferences or payor preferences when the cache has the eligibility information for the payment plan for the patient stored therein; and
returning the portion of the eligibility information to the health care service provider.
